(19) United States
(12) Patent Application Publication
(10) Pub. No.: US 2020/0174013 A1
(43) Pub. Date: Jun. 4, 2020

(54) METHOD OF DETERMINING ENDOGENOUS ANALYTE RELEASED FROM SKIN OF A SUBJECT

(71) Applicant: National Sun Yat-Sen University, Kaohsiung City (TW)

(72) Inventor: Jentaie SHIEA, Kaohsiung City (TW)

(21) Appl. No.: 16/225,350

(22) Filed: Dec. 19, 2018

(30) Foreign Application Priority Data
Nov. 30, 2018 (TW) 107143064

(51) Int. Cl. G01N 33/68; G01N 33/50; G01N 30/72

(52) U.S. Cl. CPC G01N 33/6851; H01J 49/0045; G01N 30/7233; G01N 33/5091

(57) ABSTRACT

Disclosed herein is a method of determining an endogenous analyte released from skin of a subject. The endogenous analyte is selected from the group consisting of an amino acid, a hormone, a neurotransmitter and combinations thereof. The method includes the steps of obtaining a sample which contains the endogenous analyte from the skin of the subject using a probe; desorbing the endogenous analyte from the sample; subjecting the desorbed endogenous analyte to an ionization reaction with a charged reactive species generated by an ionization device, so as to produce an ionized endogenous analyte; and determining the ionized endogenous analyte with a mass spectrometer.

### CROSS-REFERENCE TO RELATED APPLICATION

This application claims priority of Taiwanese Invention Patent Application No. 107143064, filed on Nov. 30, 2018.

### FIELD

The disclosure relates to a method of determining a biological analyte, and more particularly to a method of determining an endogenous analyte released from skin of a subject.

### BACKGROUND

Physical conditions of a subject can be reflected by the levels of various endogenous metabolites of the subject. Most newborn screening tests are conducted by needle punching on the subject to collect whole blood samples on specialized filter paper, and then measuring the concentration of amino acids and enzymes in the whole blood samples. For example, phenylketonuria, a metabolic disorder which may cause irreversible mental retardation, is commonly screened for newborn infants by measuring the concentrations of phenylalanine and tyrosine as indicators. Likewise, blood sampling is required for determining the concentrations of estrogen, the primary female sex hormone that is associated with the female menstrual cycle and menopause, and also of neurotransmitters such as serotonin and dopamine, which are associated with various mental disorders or neurological disorders.

However, blood sampling is an invasive procedure which poses risks if not conducted properly. In addition, since blood contains relatively complex components in large numbers, the blood samples needs further processing (e.g., extraction, separation, filtration and purification) prior to analysis by liquid chromatography or mass spectrometry, thereby prolonging the time for obtaining diagnosis results.

Skin is the largest organ in vertebrates, and can act as a medium for releasing several metabolites from the body to its surface. Therefore, the applicant endeavors to develop a fast analytical method to determine the endogenous metabolites released by the body to the skin, which might be helpful for screening and diagnosing diseases.

### SUMMARY

Therefore, an object of the disclosure is to provide a method of determining an endogenous analyte released from skin of a subject that can alleviate at least one of the drawbacks of the prior art.

According to this disclosure, the method includes the steps of:
obtaining a sample which contains the endogenous analyte from the skin of the subject with a probe, the endogenous analyte being selected from the group consisting of an amino acid, a hormone, a neurotransmitter and combinations thereof;
desorbing the endogenous analyte from the sample;
subjecting the desorbed endogenous analyte to an ionization reaction with a charged reactive species generated by an ionization device, so as to produce an ionized endogenous analyte; and
determining the ionized endogenous analyte with a mass spectrometer.

### BRIEF DESCRIPTION OF THE DRAWINGS

Other features and advantages of the disclosure will become apparent in the following detailed description of the embodiment (s) with reference to the accompanying drawings, of which:

FIG. 1 is a flow chart illustrating steps of an embodiment of a method of determining an endogenous analyte released from skin of a subject according to this disclosure;

FIG. 2 is a schematic view illustrating a detection system on which the method of this disclosure is performed;

FIG. 3 is multiple reaction monitoring (MRM)-mass spectra of (a) serine, (b) γ-aminobutyric acid (GABA), (c) glycine and (d) dopamine in the sample from the skin;

FIG. 4 is multiple reaction monitoring (MRM)-mass spectra of (a) epinephrine, (b) serotonin and (c) adenosine in the sample from the skin;

FIG. 5 is multiple reaction monitoring (MRM)-mass spectra of (a) histamine, (b) phenylalanine and (c) cortisol in the sample from the skin;

FIG. 6 is multiple reaction monitoring (MRM)-mass spectra of (a) valine, (b) isoleucine and leucine, (c) methionine and (d) tyrosine in the sample from the skin;

FIG. 7 is a multiple reaction monitoring (MRM)-mass spectrum of estradiol (E2) in the sample from the skin of a menstruating female; and

FIG. 8 is a mass spectrum showing a comparison of the concentration of estradiol in an adolescent male and an adolescent female within a menstrual cycle (26 days).

### DETAILED DESCRIPTION

Before the disclosure is described in greater detail, it should be noted that where considered appropriate, reference numerals or terminal portions of reference numerals have been repeated among the figures to indicate corresponding or analogous elements, which may optionally have similar characteristics.

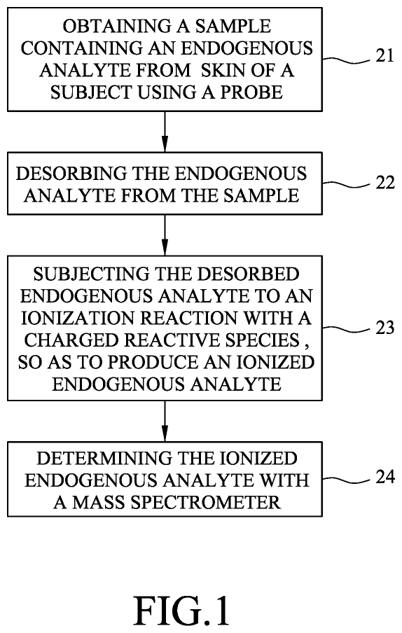

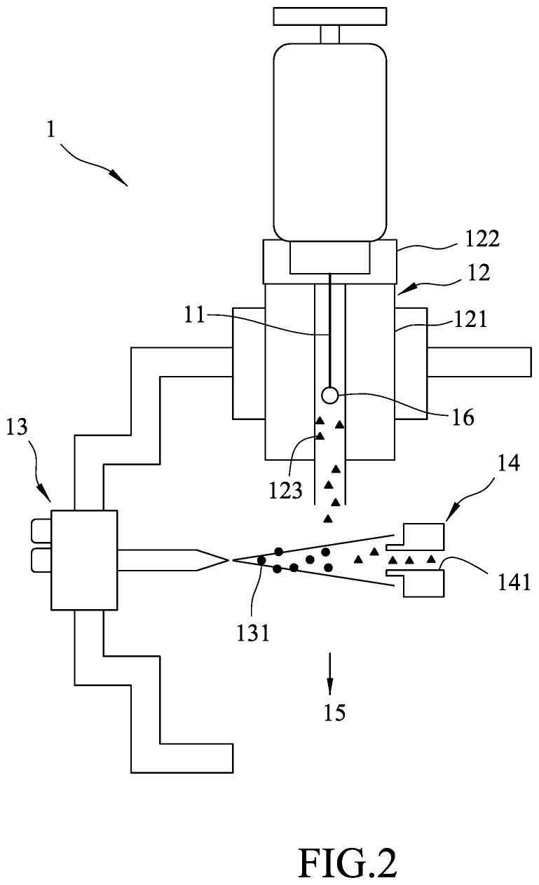

Referring to FIGS. 1 to 2, the embodiment of a method of determining an endogenous analyte released from skin of a subject according to this disclosure includes the following consecutive steps 21 to 24. The method may be conducted by virtue of a detection system 1, which includes a probe 11 for sampling, a desorption device 12, an ionizing device 13 and a mass spectrometer 14.

In step 21, a sample containing the endogenous analyte to be determined is collected using the probe 11 from the skin of the subject. Examples of the probe may include, but are not limited to, a stainless steel probe, a metal probe and a cotton swab. The sample thus obtained may be dander, sebum, epidermis, etc. The sampling site may vary based on actual requirements. For example, the common sampling site is a forehead of the subject. For detecting a special endogenous analyte that may be distributed over the skin of the entire body or in a particular body site, the sampling site would be the skin of the entire body or the particular body site.

It is noted that the skin has sebaceous glands and sweat glands, which are exocrine glands and secrete endogenous substances (such as amino acids, hormones and neurotransmitters) onto an epithelial surface. Therefore, the endogenous analyte in the sample may be an amino acid, a hormone, a neurotransmitter and combinations thereof.

Examples of the amino acid may include, but are not limited to, glutamate, aspartate, serine, γ-aminobutyric acid (GABA), glycine, valine, isoleucine, leucine, methionine, histamine and tyrosine.

Examples of the hormone may include, but are not limited to, steroid hormones, such as the sex hormones estradiol and testosterone as well as the stress hormone cortisol; eicosanoids produced by cyclooxygenases and lipoxygenases; amino acid derived-hormone, such as melatonin and thyroxine; peptide hormones, such as thyrotropin-releasing hormone and vasopressin; protein hormones, such as insulin and growth hormone; and glycoprotein hormones, such as luteinizing hormone, follicle-stimulating hormone and thyroid-stimulating hormone.

Examples of the neurotransmitter may include, but are not limited to, monoamines, such as dopamine (DA), norepinephrine (NE) (also called noradrenaline (NA)), epinephrine, histamine and serotonin (SER, 5-HT); trace amines, such as phenethylamine, N-methylphenethylamine, tyramine, 3-iodothyronamine, octopamine, tryptamine, etc.; peptides, such as oxytocin, somatostatin, substance P, cocaine and amphetamine regulated transcript, opioid peptides; purines, such as adenosine triphosphate (ATP) and adenosine; acetylcholine (ACh); and anandamide, etc.

In Step S22, the endogenous analyte is desorbed from the sample 16 by virtue of the desorption device 12. In this embodiment, the step S22 is performed with a thermal desorption device including a heating unit 121 and a gas supply unit 122. The heating unit 121 is adapted to heat the sample 16 on the probe 11, so as to desorb the thus formed endogenous analyte 123 in a gas phase. The gas supply unit 122 is adapted to provide a gas stream for carrying the desorbed endogenous analyte along a predetermined direction.

In step S23, the desorbed gas phase endogenous analyte 123 was subjected to an ionization reaction (i.e., ion-molecule reaction) with a charged reactive species 131 generated by the ionization device 13, so as to produce an ionized endogenous analyte. In this embodiment, the ionization device 13 is disposed downstream of the gas supply unit 122. The charged reactive species is moved in a direction traversing the predetermined direction 15 of the gas stream.

The ionization device 13 may include an ion source to generate the charged reactive species 131. Examples of the ion source may include, but are not limited to, an electrospray ionization (ESI) ion source, an atmospheric pressure chemical ionization (APCI) ion source, an atmospheric pressure photo ionization (APPI) ion source, a matrix assisted laser desorption ionization (MALDI) ion source, a laser desorption ionization (LDI) ion source, an atmospheric pressure ionization (API) ion source, an electron impact (EI) ion source, a chemical ionization (CI) ion source, a field ionization (FI) ion source, an inductively coupled plasma (ICP) ion source, a fast atom bombardment (FAB) ion source, a liquid secondary ion mass spectrometry (LSIMS) ion source, a laserspray ionization (LSI) ion source, a sonicspray ionization (SSI) ion source, a matrix assisted inlet ionization (MAII) ion source, and a solvent assisted inlet ionization (SAII) ion source. In this embodiment, the ion source of the ionization device 13 is an ESI ion source. In another exemplary embodiment, the ion source is an APCI ion source.

It should be noted that, the desorption device 12 used in step 22 may be a laser device to generate laser for desorbing the endogenous analyte. In this case, the ionization device 13 used in step 23 is disposed downstream of the laser.

In step 24, the ionized endogenous analyte is determined with the mass spectrometer 14. The mass spectrometer 14 has an inlet 141 for the ionized endogenous analyte passing therethrough to enter the inside of the mass spectrometer 14. In this embodiment, it may not be necessary to dispose the sample 16 in front of the inlet 141 because of the configuration of the gas supply unit 122 and the ionization device 13. Based on the mass spectrum obtained in step 24, the endogenous analyte is therefore determined.

In addition, for achieving an excellent sensitivity to the endogenous analyst that may be present in a trace amount, selected reaction monitoring (SRM), such as multiple reaction monitoring (MRM), consecutive reaction monitoring (CRM) and parallel reaction monitoring (PRM), may be applied in step 24.

According to this disclosure, the method may further include the step of removing the sample 16 from the probe 11 (e.g., burning with a flame). Therefore, the probe 11 may be repeatedly used.

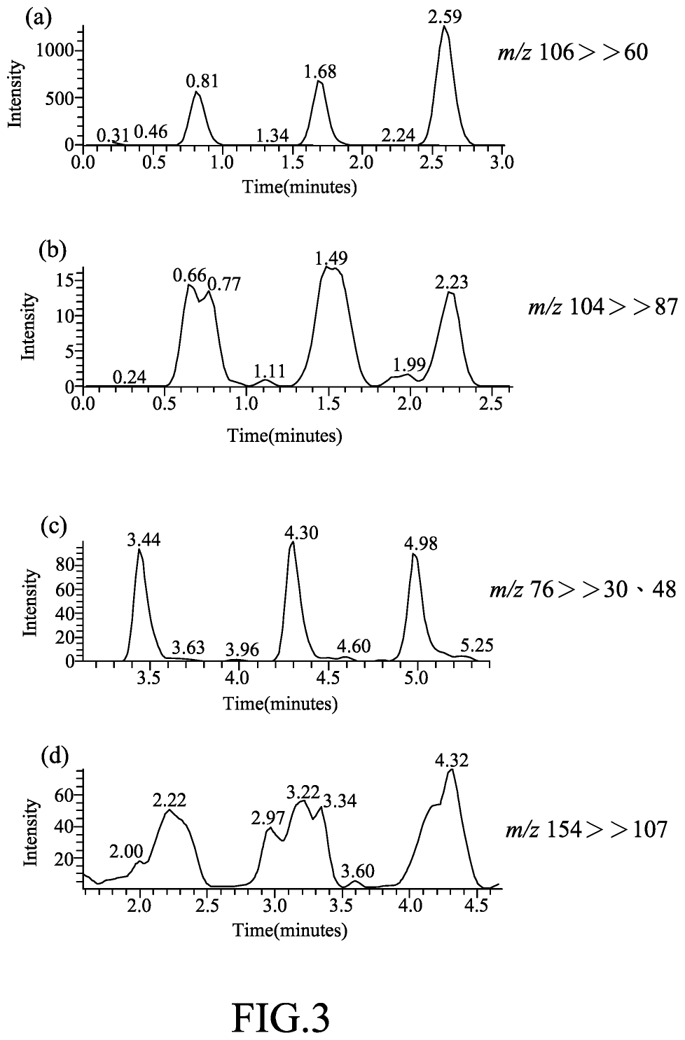

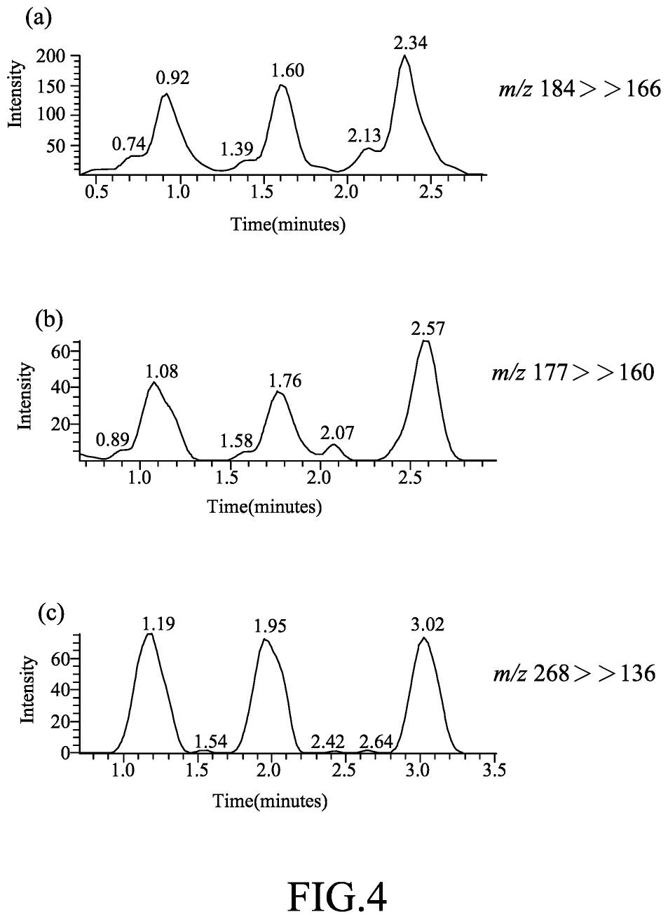

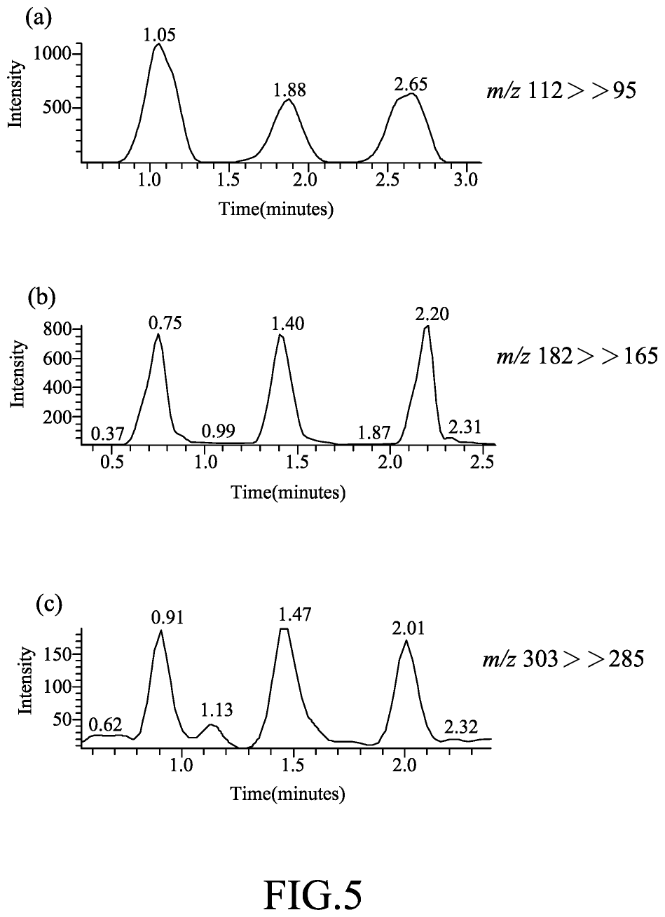

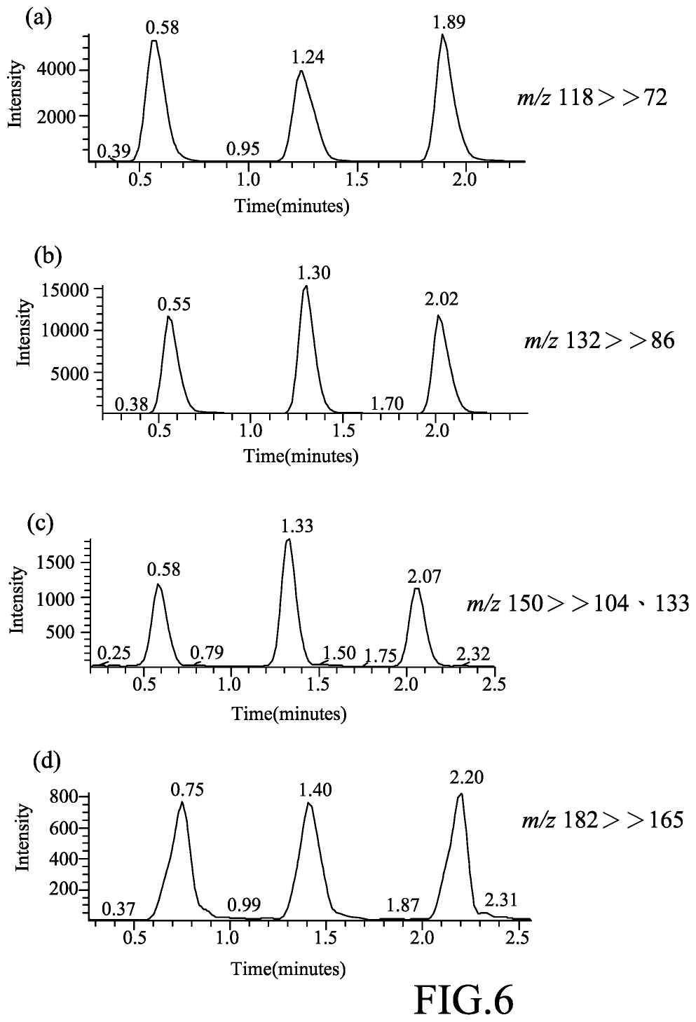

Referring to FIGS. 3 to 5, the method of this disclosure, in which thermal desorption-electrospray ionization/mass spectrometry (TD-ESI/MS) and multiple reaction monitoring (MRM) are applied, is capable of successfully detecting several endogenous amino acids and neurotransmitters released from the human skin, which includes serine, GABA, glycine, dopamine, epinephrine, serotonin, adenosine, histamine, phenylalanine and cortisol that are reported to be associated with mental disorders and neurological disorders. Other amino acids including valine, isoleucine (as well as leucine), methionine and tyrosine that serve as clinical indicators for diseases of the newborns, are also detected in the samples obtained from the skin surface by the method as mentioned above (see FIG. 6).

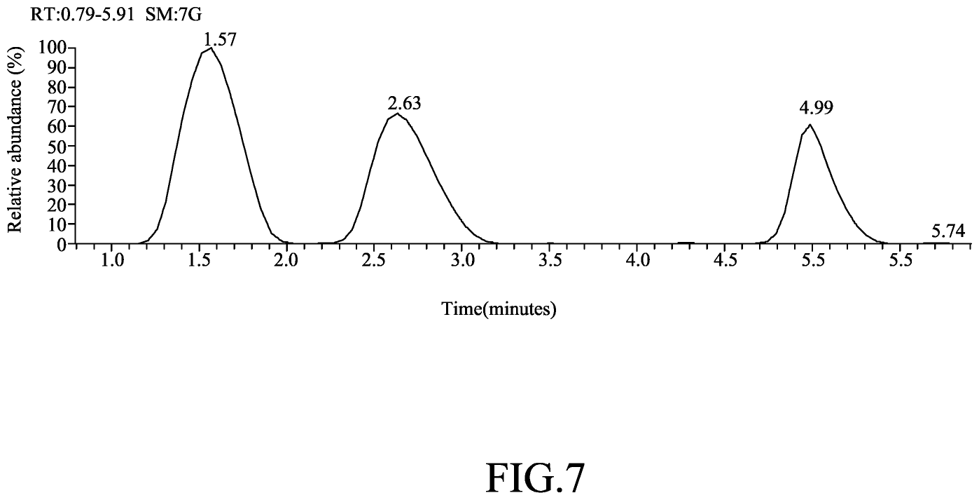

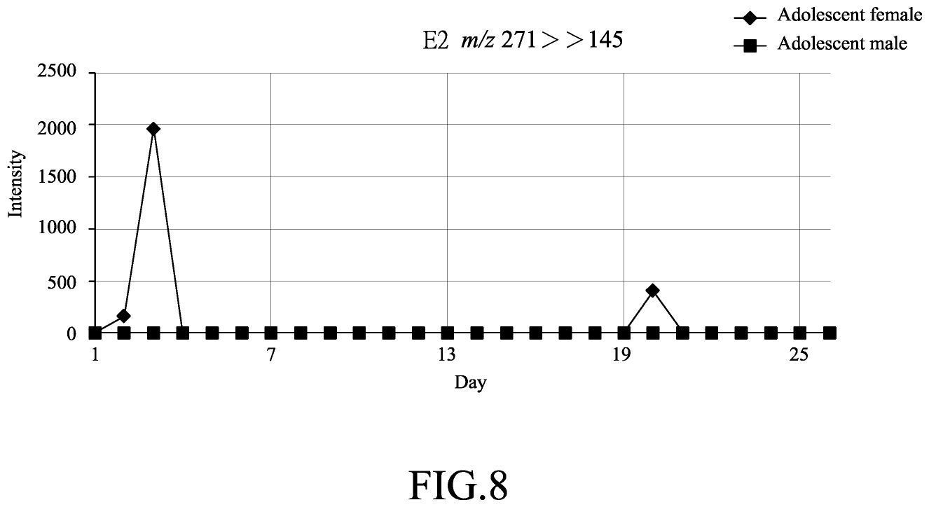

In addition, the method of this disclosure is also successfully applied in the detection of hormones, such as estradiol (E2) from the skin of a menstruating female (see FIG. 7). Moreover, FIG. 8 shows a comparison of the concentration of estradiol in an adolescent male and an adolescent female, which is monitored during a menstrual cycle (26 days) by virtue of the method of the disclosure. It can be seen from FIG. 8 that the concentration of estradiol increased sharply in the adolescent female at particular days due to the menstrual cycle. However, the adolescent male shows minimal change in the concentration of estradiol during the whole monitoring period. It should be mentioned that the concentration of estradiol in the adolescent female during remaining days of the menstrual cycle are close to that of the adolescent male, and thus are not shown in FIG. 8 for the sake of brevity.

To sum up, since using the probe to perform sampling process on the skin is non-invasive and fast, and since the sample can be directly desorbed, ionized and detected mainly under ambient conditions (1 atm) without pretreatment, the method of this disclosure thus allows direct, rapid, real-time, and high-throughput analysis of the endogenous analyte of interest in liquid or solid sample released from the skin.

In the description above, for the purposes of explanation, numerous specific details have been set forth in order to provide a thorough understanding of the embodiment. It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects, and that one or more features or specific details from one embodiment may be practiced together with one or more features or specific details from another embodiment, where appropriate, in the practice of the disclosure.

While the disclosure has been described in connection with what are considered the exemplary embodiments, it is understood that this disclosure is not limited to the disclosed embodiments but is intended to cover various arrangements included within the spirit and scope of the broadest interpretation so as to encompass all such modifications and equivalent arrangements.

What is claimed is:

1. A method of determining an endogenous analyte released from skin of a subject, comprising:
obtaining a sample which contains the endogenous analyte from the skin of the subject using a probe;
desorbing the endogenous analyte from the sample;
subjecting the desorbed endogenous analyte to an ionization reaction with a charged reactive species generated by an ionization device, so as to produce an ionized endogenous analyte; and
determining the ionized endogenous analyte with a mass spectrometer, and
wherein the endogenous analyte is selected from the group consisting of an amino acid, a hormone, a neurotransmitter and combinations thereof.

2. The method as claimed in claim 1, wherein the desorbing step is performed with a laser.

3. The method as claimed in claim 1, wherein the desorbing step is performed with a thermal desorption device, the thermal desorption device including a heating unit for heating the sample on the probe, and a gas supply unit adapted to provide a gas stream for carrying the desorbed endogenous analyte along a predetermined direction.

4. The method as claimed in claim 3, wherein the ionization device is disposed downstream of the gas supply unit.

5. The method as claimed in claim 1, wherein the ionization device includes an ion source selected from the group consisting of an electrospray ionization (ESI) ion source, an atmospheric pressure chemical ionization (APCI) ion source, an atmospheric pressure photo ionization (APPI) ion source, a matrix assisted laser desorption ionization (MALDI) ion source, a laser desorption ionization (LDI) ion source, an atmospheric pressure ionization (API) ion source, an electron impact (EI) ion source, a chemical ionization (CI) ion source, a field ionization (FI) ion source, an inductively coupled plasma (ICP) ion source, a fast atom bombardment (FAB) ion source, a liquid secondary ion mass spectrometry (LSIMS) ion source, a laserspray ionization (LSI) ion source, a sonicspray ionization (SSI) ion source, a matrix assisted inlet ionization (MAII) ion source, and a solvent assisted inlet ionization (SAII) ion source.

6. The method as claimed in claim 1, further comprising the step of removing the sample from the probe using a flame.

7. The method as claimed in claim 1, wherein the amino acid is selected from the group consisting of glutamate, aspartate, serine, γ-aminobutyric acid (GABA), glycine, valine, isoleucine, leucine, methionine, histamine, tyrosine, and combinations thereof.

8. The method as claimed in claim 1, wherein the hormone is selected from the group consisting of estradiol, testosterone, cortisol, and combinations thereof.

9. The method as claimed in claim 1, wherein the neurotransmitter is selected from the group consisting of dopamine (DA), norepinephrine (NE), epinephrine, serotonin, phenethylamine, adenosine, and combinations thereof.
